(19) United States
(12) Patent Application Publication
(10) Pub. No.: US 2008/0109082 A1
(43) Pub. Date: May 8, 2008

(54) Implant for alleviating pressure on intervertebral disks and method for restoring the height of and alleviating pressure on an intervertebral space

(76) Inventors: Ulrich Fink, Tuttlingen (DE); Charles Wing, Center Valley, PA (US)

Correspondence Address: RATNERPRESTIA, P O BOX 980, VALLEY FORGE, PA, 19482-0980, US

(21) Appl. No.: 11/973,950

(22) Filed: Oct. 11, 2007

Related U.S. Application Data
(63) Continuation of application No. PCT/EP05/04060, filed on Apr. 16, 2005.

(51) Int. Cl. A61F 2/44

(52) U.S. Cl. 623/17.16

(57) ABSTRACT

In order to improve an implant (10) for alleviating pressure on intervertebral disks, for restoring the height of and alleviating pressure on an intervertebral space of a human or animal spinal column, comprising at least two bearing elements (24, 26) for a spinous process (14, 16) each for abutting and/or securing the implant on one or two spinous processes of adjacent vertebra of the spinal column, such that as far as possible only one single operation is required to restore the height of and alleviate pressure on the intervertebral space it is suggested that the implant (10) be produced from a biocompatible, resorbable material. Furthermore, a method for restoring the height of and alleviating pressure on an intervertebral space of a human or animal spinal column is suggested.

This application is a continuation of International application No. PCT/EP2005/004060 filed on Apr. 16, 2005.

The present disclosure relates to the subject matter disclosed in International application No. PCT/EP2005/004060 of Apr. 16, 2005, which is incorporated herein by reference in its entirety and for all purposes.

### BACKGROUND OF THE INVENTION

The present invention relates to an implant for alleviating pressure on intervertebral disks, for restoring the height of and alleviating pressure on an intervertebral space of a human or animal spinal column, comprising at least two bearing elements for a spinous process each for abutting or securing the implant on one or two spinous processes of adjacent vertebra of the spinal column.

Furthermore, the present invention relates to a method for restoring the height of and alleviating pressure on an intervertebral space of a human or animal spinal column, using an implant for alleviating pressure on intervertebral disks comprising at least two bearing elements for a spinous process each for abutting and/or securing the implant on one or two spinous processes of adjacent vertebra of the spinal column.

During a prolapse of an intervertebral disk arranged between two vertebral bodies of the spinal column, nucleus material normally exits through the annulus of the intervertebral disk. One consequence of this is loss of the original height of the intervertebral disk, with the result that the bone structures of the foramen move closer and press on roots of nerve structures exiting from the spinal column. A patient often suffers from very considerable pain on account of this mechanical pressure.

If the sheath of the annulus, the so-called containment, is destroyed only in a locally limited manner, it is possible to restore the height of the nucleus and, therefore, of the entire intervertebral space again by injecting nucleus or cartilage cells into the nucleus. However, it is to be noted in this respect that the desired height is restored immediately as a result of the injection of a large cell volume but very great pressure also results in the interior of the intervertebral disk which can lead or even leads almost always to the cells dying off. This problem may be circumvented in that only a small cell volume is injected into the nucleus so that no increased pressure results. The volume within the intervertebral disk is not then increased until the extracellular matrix is formed by way of the injected cells. The disadvantage of this is that a patient can become free of pain only in the medium term or long term and several operations are very often required.

Therefore, it would be desirable to provide an implant for alleviating pressure on intervertebral disks and a method so that as far as possible only one single operation is required to restore the height of and alleviate pressure on the intervertebral space.

### SUMMARY OF THE INVENTION

In accordance with the invention, it is suggested in an implant for alleviating pressure on intervertebral disks of the type described at the outset, that the implant is produced from a biocompatible, resorbable material.

Such an implant enables the required number of cells to be injected into the nucleus in order to restore the intervertebral space to the original height with a single operation. Any pressure within the intervertebral disk will be clearly reduced by the implant for alleviating pressure on intervertebral disks and so the injected cells do not die off. Since the implant is, in addition, produced from a biocompatible, resorbable material, the pressure on the restored intervertebral disk is increased only during the course of time. Sufficient time therefore remains for an adequate stabilization of the intervertebral disk with the injected cells. As a result of a corresponding selection of the material as well as shape of the implant, it is possible for an alleviation of pressure on the intervertebral space to be reduced successively over the course of several weeks, for example, during a period of time of two weeks to 18 months. The alleviation of pressure on the intervertebral space is reduced little by little as a result of the resorbability of the implant, in contrast to which it is eliminated completely in one step in the case of a non-resorbable implant as a result of the implant being removed. Furthermore, it is of advantage when the implant is resorbable at least partially, normally completely so that no additional operation is necessary to remove the implant for alleviating pressure on intervertebral disks again. The removal of an internal fixation device for alleviating pressure on the intervertebral space can, in particular, lead to undesired trauma. In addition, the implant can also be inserted into the body by way of a minimally invasive approach when it is shaped accordingly, whereby any operation trauma is minimized and, therefore, any postoperative affect on the patient is reduced.

It is favorable when at least one of the at least two bearing elements is designed in the form of a receptacle with an insertion opening for the insertion of a spinous process in a direction parallel or transverse to a preferred direction defined by the spinous process. As a result, the introduction of the implant into the body and the abutment on the spinous process or processes is simplified, on the one hand. On the other hand, additional fixing elements for securing the implant can, in certain circumstances, be dispensed with which reduces operating time, on the one hand, and, on the other hand, does not make any additional trauma to a spinous process necessary. In addition, movement of a spinous process can be limited by way of a stop, such as that defined, for example, by a fork-like receptacle, in a desired manner.

The construction of the implant becomes particularly simple when two receptacles are provided, the insertion openings of which point away from one another. For example, the implant could be designed in the form of a double T support which defines two fork-like receptacles for the insertion of two spinous processes. In addition, the implant can be designed in a particularly slim and elongated manner as a result of such a configuration which simplifies the insertion of the implant into the body through a minimally invasive approach. Moreover, a minimal distance between the two adjacent spinous processes is predetermined by the configuration of the implant.

The receptacle is preferably of a groove-like design. Such an implant can be moved towards the spinous processes parallel to the groove-like receptacle. In addition, it is, however, also possible to insert the spinous processes into the receptacle transversely to the groove direction.

The implant will be particularly light and is, in addition, very easy to produce when it is produced entirely or partially from a plastic material. An additional advantage of producing the implant from a plastic material is that the implant can be adapted in an optimum manner to an anatomical geometry, particularly when the plastic material has rubber elastic properties and traumata can be avoided in this way.

In order to be able to predetermine a resorbability of the implant within a desired period of time, it is advantageous when the plastic material is a polymer or contains a polymer. As a consequence of the resorption, polymer chains can be successively degraded, whereby the stability of the implant as a whole is reduced in a desired manner during the course of time.

The implant is particularly biocompatible when the polymer is polylactide or contains polylactide.

Furthermore, a resorbability, in particular, of the implant may be adjusted particularly well when the plastic material is a gelatin cross-linked three dimensionally or contains a gelatin cross-linked three dimensionally.

In order to prevent the implant from becoming detached from a spinous process out of a predetermined position relative thereto, it is favorable when the implant comprises at least one securing element for securing a spinous process to a bearing element. For example, the securing element may be a fixing element, with which the bearing element can be secured to a spinous process. It would, however, also be conceivable to provide a securing element which restricts the freedom of movement of the implant relative to a spinous process only conditionally, for example, by reducing the number of degrees of freedom of movement of the implant relative to a spinous process from two to one.

So that a spinous process cannot exit again from the receptacle through the insertion opening, it is advantageous when the insertion opening can be closed or locked.

A closure element is favorably provided for closing the insertion opening. The closure element can be an individual component or, however, also be designed in one piece with the rest of the implant.

So that the insertion opening can be closed in a simple manner, it is advantageous when the closure element is mounted on the receptacle so as to be movable. For example, it can be mounted, for example, via a film hinge such that it cannot become detached from the implant unintentionally.

The insertion opening may be closed in a particularly simple manner when the closure element is pivotally or displaceably mounted. In this way, the insertion opening can be closed, in particular, as a result of simple pivoting of the closure element, for example, after the implant has been inserted and abutted on a spinous process.

In accordance with one preferred embodiment of the invention, it may be provided for the closure element to be lockable or connectable in a snap-in manner to the receptacle in a closure position, in which the insertion opening is closed. It is thus possible in a simple manner to prevent the closure element from releasing the insertion opening unintentionally.

In order to be able to use the implant universally, in particular, for patients of different sizes, it is favorable when a distance between the at least two bearing elements is variable. This makes it possible to adapt the implant individually to a patient and also selectively predetermine a desired alleviation of pressure on the intervertebral space.

So that the bearing elements maintain a set distance between them, it is advantageous when the two bearing elements can be secured relative to one another at a predetermined distance.

The distance between the two bearing elements is advantageously variable in discrete steps. As a result, the construction of the implant is simplified, on the one hand, for example, by providing locking strips or teeth movable relative to one another; on the other hand, the handling capability as well as the stability of the implant are also improved.

In principle, it would be conceivable to design the implant in one piece. It is, however, favorable when the implant comprises at least two implant parts each having at least one bearing element and when the two implant parts can be secured to one another. As a result, a relative arrangement of the bearing elements can, for example, be determined individually and differently depending on the requirements of each patient. Moreover, a distance of the bearing elements from one another and, therefore, an alleviation of pressure on the intervertebral space can also be predetermined in a desired manner.

The two implant parts can preferably be secured relative to one another in different positions. This may be possible in the form of not only discrete but also non-discrete positions.

So that the implant can be secured to a spinous process in a reliable manner, it is favorable when the implant comprises at least one fixing element for securing the at least one bearing element to a spinous process.

In order to increase the stability of the implant and, in addition, ensure that the implant cannot become detached from a spinous process, it is advantageous when the implant has at least one fixing element receptacle for the at least one fixing element, that the fixing element can be driven into the spinous process or be secured to it and that the fixing element is held in the fixing element receptacle. For example, a bone pin or a bone screw could be guided through a bore in the implant forming a fixing element receptacle and be driven into the bone. Alternatively, a thread could also be guided through a bore and wound around the spinous process. It would, however, also be conceivable to design the fixing element in the form of a plug or a rivet.

In order to increase the stability of a connection between the implant and a spinous process, it is favorable when the at least one fixing element can be secured to the receptacle passing transversely through it.

Four fixing elements per bearing element are advantageously provided. As a result, a particularly secure connection between the implant and the spinous process can be achieved.

In accordance with one preferred embodiment of the invention, it may be provided for the at least one fixing element to be a bone pin, a bone screw or a thread. With such fixing elements, the implant may be secured to a spinous process in a simple manner. The at least one fixing element can, in addition, like the entire implant, be produced from a resorbable material, for example, from the same material as the rest of the implant. It would, however, also be conceivable to provide fixing elements which are produced from a material which is biocompatible but not resorbable.

Further, it is suggested in accordance with the invention, in a method of the type described at the outset, that the implant is produced from a biocompatible, resorbable material.

It is possible with the method according to the invention to restore the height of the intervertebral space, for example, due to injection of a cell volume required for this purpose into the nucleus of the intervertebral disk in a single surgical operation without having to run the risk of the injected cells dying off on account of an increased internal pressure in the nucleus. Furthermore, the suggested procedure allows an implant to be selected which decreases its pressure alleviating function little by little during a specific period of time.

It is advantageous when at least one of the at least two bearing elements is designed in the form of a receptacle with an insertion opening for the insertion of a spinous process in a direction parallel or transverse to a preferred direction defined by the spinous process. As a result, the operation can be carried out considerably more quickly since a spinous process can be introduced into the receptacle in a simple manner.

Two receptacles are preferably provided, the insertion openings of which point away from one another. As a result, it is possible to prevent spinous processes inserted into the receptacles from being held at a minimal distance from one another.

A spinous process can be inserted into the receptacle particularly easily when this is of a groove-like design.

An implant can be introduced into a human body through a minimally invasive approach particularly easily when the implant is produced entirely or partially from a plastic material; for example, this plastic material can have a certain elasticity so that the implant can be pressed together somewhat during insertion into the body.

A plastic material is preferably used which is a polymer or contains a polymer. A resorption time of the implant can be predetermined in a desired manner as a result.

An implant will be particularly biocompatible when a polymer is used which is polylactide or contains polylactide.

A plastic material is preferably used which is a gelatin cross-linked three dimensionally or contains a gelatin cross-linked three dimensionally. The use of gelatin as material for the production of the implant enables a desired resorption time of the implant and, therefore, a successive alleviation of pressure on the intervertebral space during a specific period of time to be predetermined by adjusting a degree of cross-linking of the gelatin.

In order to prevent the implant being able to detach from a spinous process in an undesired manner, it is favorable when the implant comprises at least one securing element and when a bearing element is secured to a spinous process with the at least one securing element. For example, a fixing element already described above in greater detail can be used as securing element. Alternatively, it would also be conceivable to provide a closure element for the insertion opening, whereby a spinous process is prevented from being able to exit from the receptacle.

In order to prevent a spinous process from exiting from the receptacle, it is advantageous when the insertion opening is closed once the spinous process has been inserted into the receptacle.

The insertion opening may be closed particularly easily when it is closed with a closure element. A closure element specially provided for the closure of the insertion opening helps to ensure that the length of an operation is minimized.

The insertion opening may be closed with the closure element particularly easily when the closure element is mounted on the receptacle so as to be movable. After insertion of the spinous process into the receptacle, the closure element need then be moved, for example, by a surgeon only from an insertion position, in which the insertion opening is free, into a closure position, in which the insertion opening is closed.

The closure element may be transferred into the closure position in a particularly fast and simple manner when the closure element is pivotally or displaceably mounted.

So that a secure connection between the implant and the spinous process can be brought about, it is advantageous when the closure element is locked or connected in a snap-in manner to the receptacle in a closure position, in which the insertion opening is closed.

A distance between the at least two bearing elements is favorably altered in a desired manner prior to or following the insertion of the implant. In this way, the implant can be adapted individually to the respective patient, in particular, to situations, in which distances between spinous processes vary considerably.

In order to prevent a distance between the two bearing elements from being able to change after insertion into the human body and termination of the operation, it is favorable when the two bearing elements are secured relative to one another at a predetermined distance prior to or following the insertion of the implant.

For a surgeon, a distance between the two bearing elements may be brought about in an advantageous manner without additional tools when, for example, the distance between the two bearing elements is altered in discrete steps prior to or following the insertion of the implant. For example, this can be brought about via the provision of locking strips which can be displaced relative to one another or corresponding teeth.

In order to make an individual adaptation of the implant to a patient possible, it is favorable when the implant comprises at least two implant parts each having at least one bearing element and when the two implant parts are secured to one another prior to or following the insertion of the implant. As a result, a distance between the two bearing elements can, for example, be adjusted and, therefore, a desired alleviation of pressure on the intervertebral space predetermined.

The two implant parts can advantageously be secured relative to one another in different positions and are secured to one another in a predetermined position. As a result, the implant can be prepared during the operation in accordance with the requirements on account of an orthopedic situation.

It is advantageous when the implant comprises at least one fixing element and when the at least one bearing element is secured to a spinous process with the at least one fixing element. The implant may thus be secured to a spinous process in a simple and reliable manner.

It is favorable when the implant has at least one fixing element receptacle for the at least one fixing element and when the fixing element is inserted into the fixing element receptacle and driven into the spinous process or secured to it. For example, the implant can be held securely on a spinous process as a result of a bone pin being knocked in or a bone screw screwed in and prior guidance of these fixing elements through a corresponding fixing element receptacle of the implant, for example, a bore. Alternatively, the implant can also be secured to a spinous process with a thread which has been guided through a bore, wound around the spinous process and tied to it.

In accordance with a preferred variation of the method according to the invention, it may be provided for the at least one fixing element to be secured to the receptacle passing transversely through it. A connection between the implant and the spinous process is thus secured in a particularly good manner. In particular, it may also be provided for the spinous process to likewise be provided with a corresponding receptacle for the fixing element, for example, one or more bores prior to the insertion of the fixing element.

In order to ensure a particularly good securing of the implant on a spinous process, four fixing elements are used per bearing element.

The method may be carried out particularly easily and, therefore, the implant secured to a spinous process when a bone pin, a bone screw or a thread is used as fixing element and when the bearing element is securely pinned, securely screwed or securely tied to a spinous process.

In accordance with a further, preferred variation of the method according to the invention, it may be provided for a minimally invasive approach to the human or animal body to be opened, for the implant to be guided to the spinous processes of the vertebra through the minimally invasive approach and for cartilage cells to also be injected into a nucleus of a deformed or damaged intervertebral disk during this operation. This procedure makes it possible for the intervertebral space to be adjusted to its original height and alleviated of pressure with the implant with only one surgical operation and simultaneous minimalizing of operation trauma.

An implant is preferably used which is completely resorbed in a period of three weeks to six months. In this way, the injected cells are given sufficient time to restore the stability of the intervertebral disk in a desired manner.

The following description of preferred embodiments of the invention serves to explain the invention in greater detail in conjunction with the drawings.

### BRIEF DESCRIPTION OF THE DRAWINGS

FIG. 1: shows a first embodiment of an implant according to the invention secured to spinous processes of a spinal column;

FIG. 2: shows an enlarged view of the implant from FIG. 1;

FIG. 3: shows a perspective view of a second embodiment of an implant according to the invention; and

FIG. 4: shows a perspective view of a third embodiment of an implant according to the invention.

### DETAILED DESCRIPTION OF THE INVENTION

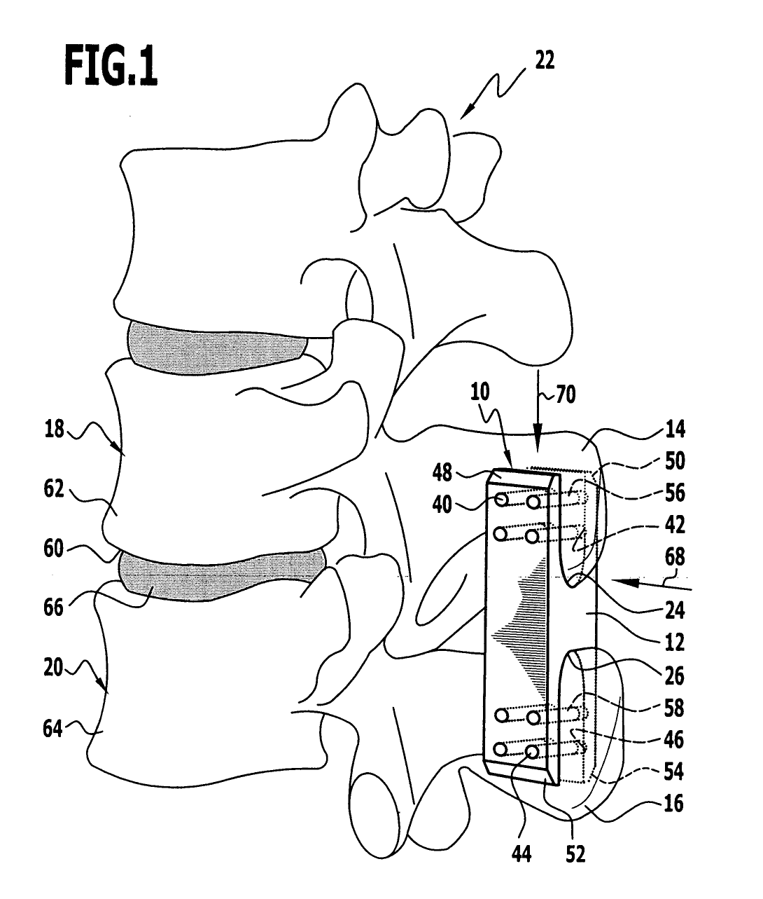

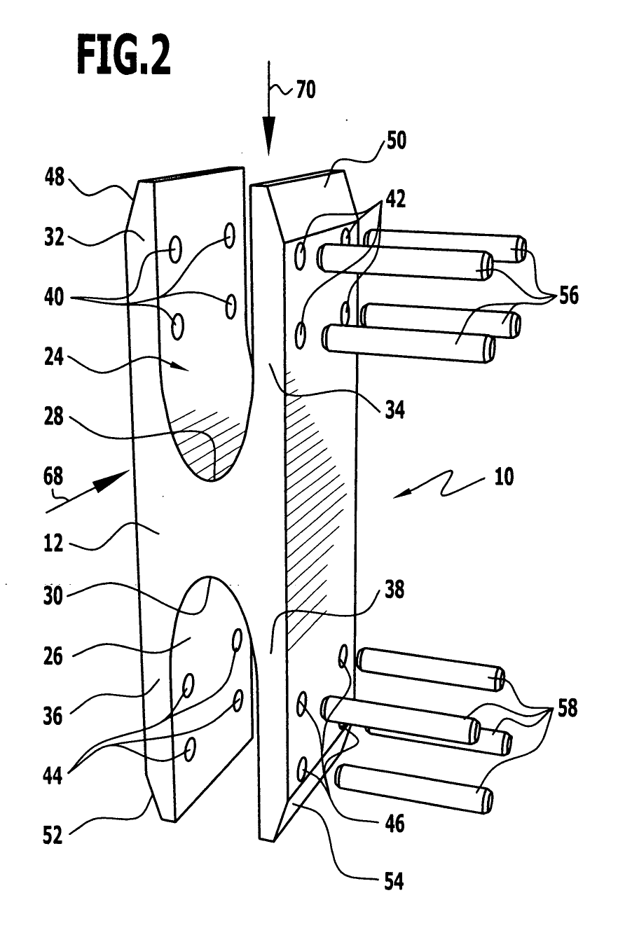

An implant 10 for alleviating pressure on intervertebral disks according to the invention and provided with the reference numeral 10 is illustrated in FIGS. 1 and 2 and this implant will be designated in the following only as implant for the sake of simplicity.

The implant 10 is formed by an elongated, basic member 12 which is essentially in the shape of a parallelepiped and has an approximately square cross section. Proceeding from oppositely located end faces of the basic member 12, two groove-like receptacles, which point away from one another, are provided for spinous processes 14 and 16, respectively, of adjacent vertebra 18 and 22 of a spinal column 22 in the form of groove-like recesses 24 and 26. The recesses 24 and 26 are each limited by a curved groove base 28 and 30, respectively, as well as two wall sections 32 and 34 or 36 and 38, respectively, which extend parallel to one another away from the groove base 28 and 30, respectively, and are aligned parallel to one another. Four transverse bores 40 and 42, which are aligned coaxially to one another and approximately define a square, are provided in each of the wall sections 32 and 34. In an analogous way, four transverse bores 44 and 46, which are aligned coaxially to one another, are also provided in each of the wall sections 36 and 38. The wall sections 32, 34, 36 and 38 are at somewhat of an angle on their outer sides proceeding from their free ends so that a wall thickness increases continuously proceeding from their respectively free ends up to a maximum wall thickness and, therefore, inclined surface sections 48, 50, 52 and 54 are formed.

In the same way as the transverse bores 40, 42, 44 and 46 can be provided in the wall sections 32, 34, 36 and 38 only optionally or in a small number or also only in one of the two recesses 24 and 26, the implant 10 also comprises only optionally a total of eight identical, essentially cylindrical locking pins, namely four locking pins 56 and four locking pins 58, respectively. An external diameter of the locking pins 56 and 58 is adapted to an internal diameter of the transverse bores 40 and 42 or 44 and 46, respectively, so that the locking pins 56 and 58 can be pushed into the transverse bores 40 and 42 or 44 and 46, respectively, and held in a clamped manner in them.

Both the basic member 12 and the locking pins 56 and 58 are preferably produced from a resorbable plastic material, for example, a polymer such as polylactide.

The operative procedure for restoring the height of an intervertebral space 60 formed between two vertebral bodies 62 and 64 will be explained in greater detail in the following in conjunction with FIG. 1.

As a result of a prolapse of an intervertebral disk 66 arranged in the intervertebral space 60 between the vertebral bodies 62 and 64, nucleus material can exit through the annulus of the intervertebral disk 66. As a result, a distance between the intervertebral bodies 62 and 64 is reduced. In order to compensate for this loss in height, a cell volume sufficient to restore the height of the intervertebral disk 66 is injected into the annulus of the spinal column 22 alleviated of pressure after a minimally invasive approach has been opened. Subsequently or also already prior to injection of the cells, the implant 10 is inserted through the minimally invasive approach and pushed between the spinous processes 14 and 16 proceeding from the dorsal side, i.e., in the direction of arrow 68. Alternatively thereto, it would also be conceivable to push each of the spinous processes 14 and 16 into the recesses 24 and 26, respectively, transversely to a direction defined by the spinous processes which extends essentially parallel to a direction specified by the arrow 68, i.e., the spinous process 14, for example, into the recess 24 in the direction of arrow 70.

In a next step, if the implant has transverse bores 40, 42, 44 and 46 in the sections 32, 34, 36 or 38, transverse bores can be optionally made in the spinous processes 14 and 16 coaxially to the transverse bores 40, 42, 44 and 46 so that the locking pins 56 and 58 can be pushed not only through the transverse bores 40, 42, 44 and 46 but also through the transverse bores now provided in the spinous processes 14 and 16. The implant 10 can be secured in the manner described to one or also to both spinous processes 14 and 16, respectively. It is ensured by this securing in place that the implant 10 cannot become detached from the respective spinous process 14 or 16 secured thereto. Without the securing in place with the locking pins 56 and 58 as described, only movement of the two spinous processes 14 and 16 towards one another would be limited but not movement of the two spinous processes 14 and 16 away from one another.

The intervertebral disk 66 restored to its height again by the injection of cells is alleviated of pressure by the implant 10 and so the pressure in the intervertebral disk 66 cannot become so great that the injected cells die off.

Following the optional securing in place of the implant 10 in the spinous processes 14 and 16, the minimally invasive approach can be closed again. The body of the patient can, of course, also be opened with a larger approach for carrying out the operation described.

The movability of the intervertebral space 60 is limited by the implant 10 until the implant 10 is resorbed to such an extent that it can be destroyed by forces acting on the implant 10 on account of movement of the spinal column 22. Until this point of time of the resorption of the implant 10, the injected cells have the possibility of filling out the intervertebral space 60 with load-resistant cartilage tissue.

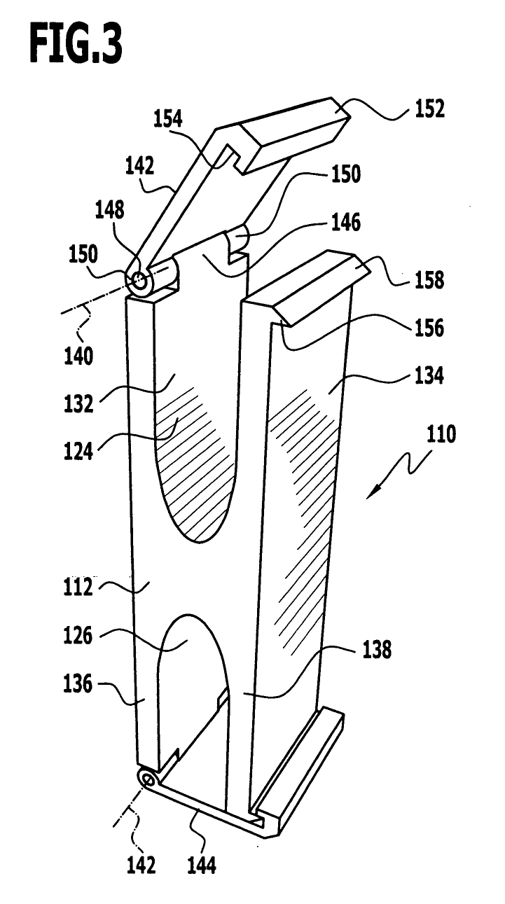

A second implant provided altogether with the reference numeral 110 will be described in greater detail in the following in conjunction with FIG. 3. It has a basic member 112 which is formed in an analogous way to the basic member 12 and is provided with recesses 124 and 126 which correspond in their shape to the recesses 24 and 26.

The wall sections 132, 134 or 136 and 138, respectively, of the recesses 124 and 126 are not, however, provided with transverse bores. By comparison, two covers 142 and 144 are mounted on free ends of the wall sections 132 and 136 so as to be pivotable about pivot axes 140 and 142, respectively, which extend parallel to one another. The covers 142 and 144 are of an identical design and so their mounting will be described in greater detail in the following only in conjunction with the cover 142.

The free end of the wall section 132 supports a bearing bracket 146 which is bored through parallel to the pivot axis 140 and through which a bearing shaft 148 extends. Free ends of the bearing shaft 148 projecting out of the bearing bracket 146 on both sides engage in bearing rings 150 of the cover 142 which border on the bearing bracket 146 on both sides so that the cover 142 can be pivoted relative to the base member 112 about the pivot axis defined by the bearing shaft 148. A free end of the cover 142 supports a snap-in projection 152 which has a locking groove 154 pointing in the direction towards the pivot axis 140. A snap-in nose 156 with an inclined slide-on surface 158 is integrally formed on the wall section 134 on an outer side, pointing away from the pivot axis 140.

If the free end of the cover 142 is moved in the direction towards the snap-in nose 156, an outer surface of the snap-in projection 152 slides first of all along the slide-on surface 158, whereby the cover 142 is deformed somewhat. If the cover 142 is pivoted further, the snap-in nose 156 snaps into the locking groove 154, whereby the recess 124 is closed on the end side. FIG. 3 illustrates at the bottom how the cover 144 closes the recess 126 in the manner described.

The implant 110 is inserted between the spinous processes 14 and 16 in a similar manner to the implant 10. The recesses 124 and 126 are dimensioned such that the spinous processes 14 and 16 can be introduced into them and secured in them as a result of the end-side closure of the recesses 124 and 126. The covers 142 and 144 can be closed either prior to or following the insertion of the spinous processes 14 and 16, respectively.

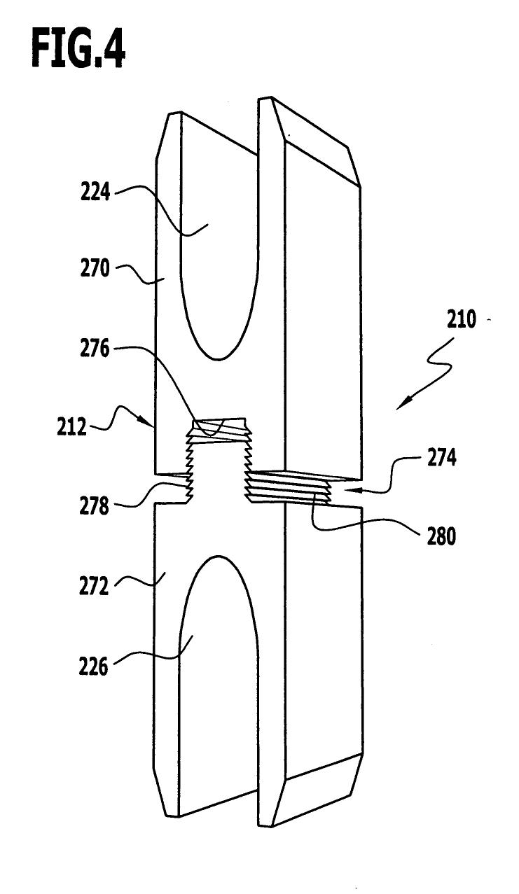

A third embodiment of an implant provided altogether with the reference numeral 210 is illustrated in FIG. 4. It corresponds essentially to the implant 10 in its basic form. However, the implant 210 is designed in two parts, i.e., the basic member 212 is divided into an upper part 270 and a lower part 272. The upper part 270 has a recess 224 corresponding to the recess 24, the lower part 272 has a recess 226 corresponding to the recess 26.

In order to connect the two parts 270 and 272, a snap-in connection 274 is provided which comprises a toothed groove 276 and a projection 278 which can be inserted into the toothed groove. The groove 276 is provided so as to extend parallel to the recess 224 on the upper part 270. The projection 278, on the other hand, projects away from the recess 226 on the lower part 272. Side surfaces of the groove 276 as well as the projection 278 which abut on one another are provided with rows of teeth 280 which correspond to one another and allow movement of the two parts 270 and 272 towards one another as a result of individual teeth of the rows of teeth 280 sliding along one another but not any movement of the parts 270 and 272 away from one another.

A distance between the recesses 224 and 226 can be altered prior to the implant 210 being introduced into the body or even after its introduction. In the case of the embodiment described, it is possible to increase the distance between the recesses 224 and 226 only with difficulty. Therefore, the implant 210 is preferably introduced into the body of the patient with a maximum possible distance between the recesses 224 and 226 and the distance reduced as required by way of movement of the two parts 270 and 272 towards one another.

One or more transverse bores may be optionally provided in the implant 210, as in the case of the implant 10, or one or two covers, as in the case of the implant 110.

1. Implant for alleviating pressure on intervertebral disks, for restoring the height of and alleviating pressure on an intervertebral space of a human or animal spinal column, comprising at least two bearing elements for a spinous process each for abutting and/or securing the implant on one or two spinous processes of adjacent vertebra of the spinal column, wherein the implant is produced from a biocompatible, resorbable material.

2. Implant as defined in claim 1, wherein at least one of the at least two bearing elements is designed in the form of a receptacle with an insertion opening for the insertion of a spinous process in a direction parallel or transverse to a preferred direction defined by the spinous process.

3. Implant as defined in claim 2, wherein two receptacles are provided, the insertion openings thereof pointing away from one another.

4. Implant as defined in claim 2, wherein the receptacle is of a groove-like design.

5. Implant as defined in claim 1, wherein the implant is produced entirely or partially from a plastic material.

6. Implant as defined in claim 5, wherein the plastic material is a polymer or contains a polymer.

7. Implant as defined in claim 6, wherein the polymer is polylactide or contains polylactide.

8. Implant as defined in claim 6, wherein the plastic material is a gelatin cross-linked three dimensionally or contains a gelatin cross-linked three dimensionally.

9. Implant as defined in claim 1, wherein the implant comprises at least one securing element for securing a spinous process to a bearing element.

10. Implant as defined in claim 2, wherein the insertion opening is closable.

11. Implant as defined in claim 2, wherein a closure element for closing the insertion opening is provided.

12. Implant as defined in claim 11, wherein the closure element is mounted on the receptacle so as to be movable.

13. Implant as defined in claim 12, wherein the closure element is pivotally or displaceably mounted.

14. Implant as defined in claim 11, wherein the closure element is lockable or connectable in a snap-in manner to the receptacle in a closure position, the insertion opening being closed in said position.

15. Implant as defined in claim 1, wherein a distance between the at least two bearing elements is variable.

16. Implant as defined in claim 15, wherein the two bearing elements are securable relative to one another at a predetermined distance.

17. Implant as defined in claim 15, wherein the distance between the two bearing elements is variable in discrete steps.

18. Implant as defined in claim 1, wherein the implant comprises at least two implant parts each having at least one bearing element and wherein the two implant parts are securable to one another.

19. Implant as defined in claim 18, wherein the two implant parts are securable relative to one another in different positions.

20. Implant as defined in claim 1, wherein the implant comprises at least one fixing element for securing the at least one bearing element to a spinous process.

21. Implant as defined in claim 20, wherein the implant has at least one fixing element receptacle for the at least one fixing element, wherein the fixing element is adapted to be driven into the spinous process or secured to it and wherein the fixing element is held in the fixing element receptacle.

22. Implant as defined in claim 20, wherein the at least one fixing element is securable to the receptacle passing transversely through it.

23. Implant as defined in claim 20, wherein four fixing elements are provided per bearing element.

24. Implant as defined in claim 20, wherein the at least one fixing element is a bone pin, a bone screw or a thread.

25. Method for restoring the height of and alleviating pressure on an intervertebral space of a human or animal spinal column, using an implant for alleviating pressure on intervertebral disks with at least two bearing elements for a spinous process each for abutting and/or securing the implant to one or two spinous processes of adjacent vertebra of the spinal column, wherein the implant is produced from a biocompatible, resorbable material.

26. Method as defined in claim 25, wherein at least one of the at least two bearing elements is designed in the form of a receptacle with an insertion opening for the insertion of a spinous process in a direction parallel or transverse to a preferred direction defined by the spinous process.

27. Method as defined in claim 26, wherein two receptacles are provided, the insertion openings thereof pointing away from one another.

28. Method as defined in claim 26, wherein the receptacle is of a groove-like design.

29. Method as defined in claim 25, wherein the implant is produced entirely or partially from a plastic material.

30. Method as defined in claim 29, wherein the plastic material used is a polymer or contains a polymer.

31. Method as defined in claim 30, wherein the polymer used is polylactide or contains polylactide.

32. Method as defined in claim 30, wherein the plastic material used is a gelatin cross-linked three dimensionally or contains a gelatin cross-linked three dimensionally.

33. Method as defined in claim 25, wherein the implant comprises at least one securing element and wherein a bearing element is secured to a spinous process with the at least one securing element.

34. Method as defined in claim 26, wherein the insertion opening is closed once the spinous process has been inserted into the receptacle.

35. Method as defined in claim 26, wherein the insertion opening is closed by a closure element.

36. Method as defined in claim 35, wherein the closure element is mounted on the receptacle so as to be movable.

37. Method as defined in claim 36, wherein the closure element is pivotally or displaceably mounted.

38. Method as defined in claim 35, wherein the closure element is locked or connected in a snap-in manner to the receptacle in a closure position, the insertion opening being closed in said position.

39. Method as defined in claim 25, wherein a distance between the at least two bearing elements is altered in a desired manner prior to or following the insertion of the implant.

40. Method as defined in claim 39, wherein the two bearing elements are secured relative to one another at a predetermined distance prior to or following the insertion of the implant.

41. Method as defined in claim 39, wherein the distance between the two bearing elements is altered in discrete steps prior to or following the insertion of the implant.

42. Method as defined in claim 25, wherein the implant comprises at least two implant parts each having at least one bearing element and wherein the two implant parts are secured to one another prior to or following the insertion of the implant.

43. Method as defined in claim 42, wherein the two implant parts are securable relative to one another in different positions and are secured to one another in a predetermined position.

44. Method as defined in claim 25, wherein the implant comprises at least one fixing element and wherein the at least one bearing element is secured to a spinous process with the at least one fixing element.

45. Method as defined in claim 44, wherein the implant has at least one fixing element receptacle for the at least one fixing element and wherein the fixing element is inserted into the fixing element receptacle and driven into the spinous process or secured to it.

46. Method as defined in claim 44, wherein the at least one fixing element is secured to the receptacle passing transversely through it.

47. Method as defined in claim 44, wherein four fixing elements are used per bearing element.

48. Method as defined in claim 44, wherein a bone pin, a bone screw or a thread is used as fixing element and wherein the bearing element is securely pinned, securely screwed or securely sewn to a spinous process.

49. Method as defined in claim 25, wherein a minimally invasive approach to the human or animal body is opened, wherein the implant is guided to the spinous processes of the vertebra through the minimally invasive approach and wherein during this operation cartilage cells are also injected into a nucleus of a deformed or damaged intervertebral disk.

50. Method as defined in claim 25, wherein the implant used is completely resorbed in a period of time of 3 weeks to 6 months.
